(19) United States
(12) Patent Application Publication
(10) Pub. No.: US 2026/0265788 A1
(43) Pub. Date: Sep. 10, 2026

(54) CARBON DIOXIDE FIXATION STRAIN AND METHOD FOR PRODUCING METABOLITES USING SAME

(71) Applicant: KOREA INSTITUTE OF SCIENCE AND TECHNOLOGY, Seoul (KR)

(72) Inventors: Youngsoon UM, Seoul (KR); Sun Mi LEE, Seoul (KR); Gyeongtaek GONG, Seoul (KR); Ja Kyong KO, Seoul (KR); Jung Ho AHN, Seoul (KR); Taekwan MOON, Seoul (KR)

(21) Appl. No.: 19/166,420

(22) PCT Filed: Apr. 12, 2024
(86) PCT No.: PCT/KR2024/095695

§ 371 Date: Sep. 17, 2025

(30) Foreign Application Priority Data
Apr. 13, 2023 (KR) 10-2023-0048748
Apr. 9, 2024 (KR) 10-2024-0048094

(51) Int. Cl. C12P 7/06; C12N 1/20; C12P 7/04; C12P 7/16; C12P 7/40; C12P 7/52; C12P 7/54

(52) U.S. Cl. CPC C12P 7/065; C12N 1/20; C12P 7/04; C12P 7/16; C12P 7/40; C12P 7/52; C12P 7/54

(57) ABSTRACT

The present disclosure discloses a novel Clostridium sp. strain having improved carbon dioxide fixation ability, a method for producing biochemicals from carbon dioxide using the novel strain, and a method for fixing carbon dioxide using the novel strain. The novel strain, according to the present disclosure, has a significantly improved carbon dioxide utilization ability compared to conventional acetogen strains, and thus, it is possible to efficiently produce biochemicals derived from carbon dioxide through fermentation of the new strain.

### CROSS-REFERENCE TO RELATED APPLICATIONS

The present application is the National Stage filing under 35 U.S.C. 371 of International Application No. PCT/KR2024/095695, filed on Apr. 12, 2024, which claims priority to and the benefit of Korean Patent Application Nos. 10-2023-0048748 and 10-2024-0048094 filed in the Korean Intellectual Property Office on Apr. 13, 2023 and Apr. 9, 2024, respectively, the entire contents of which are incorporated herein by reference.

### TECHNICAL FIELD

The present specification discloses a novel acetogen strain having improved carbon dioxide utilization ability and a method for producing metabolites using the same.

### DESCRIPTION ABOUT NATIONAL SUPPORT RESEARCH AND DEVELOPMENT

This research was conducted through the following national projects.
Department name: Ministry of Science and ICT, Name of project management (professional) institution: Korea Institute of Science and Technology, Research project name: Research operating expense support of the Korea Institute of Science and Technology (major project expense), Research title: Development of e-chemical manufacturing technology, Project number: 2E31830, Project specific number: 1711173304
Department name: Ministry of Economy and Finance, Name of project management (professional) institution: National Research Foundation of Korea, Research project name: Development of biodegradable bioplastics based on biomass for carbon neutral, Research title: Development of technology for the production of 100% biomass-based bioplasticizers replacing petroleum-based plasticizers, Project number: 2022M3J4A1052750, Project specific number: 1055000966

### BACKGROUND ART

When most chemical compounds used in industry are produced, large amounts of fossil fuels are used. The use of fossil fuels generates greenhouse gases, which are the primary cause of global warming, and the problems caused by climate change resulting from global warming are getting worse with each passing generation. A carbon capture, utilization and storage (CCUS) technology is a technology that captures, uses and stores carbon dioxide, which accounts for the majority (91%) of greenhouse gases, and since the biological conversion of carbon dioxide can reduce greenhouse gases and produce biofuels at the same time through biological conversion using carbon dioxide as a carbon source, technologies for using carbon dioxide as an energy source to replace fossil fuels have been studied in various industrial fields.

Acetogen microorganisms are microorganisms that have the Wood-Ljungdahl pathway, and thus fix carbon dioxide using hydrogen as an electron donor or grow using a syngas comprising carbon monoxide/hydrogen/carbon dioxide and industrial waste gas, and produce acetyl-CoA, and research for using the microorganisms to produce biocompounds has been actively conducted. However, most acetogen microorganisms are limited in their production of C2 organic acids and alcohols, such as acetic acid and ethanol. A Clostridium sp. JS66 strain is, as an acetogen, a strain capable of producing high-value-added multicarbon compounds such as short-chain and medium-chain fatty acids (C2-C6) and alcohols as metabolites using various sugar sources and C1 gases derived from industrial waste gas as carbon sources. However, since the wild-type Clostridium sp. JS66 strain has a slow growth rate and has low productivity of metabolites under conditions where carbon dioxide is a sole carbon source due to its low carbon dioxide utilization ability compared to carbon monoxide, the strain is limited in its industrial use.

### DISCLOSURE

### Technical Problem

In an aspect, a problem to be solved by the present disclosure is to provide a strain having improved carbon dioxide utilization ability by solving the slow growth rate and carbon dioxide consumption rate caused by the poor carbon dioxide utilization ability of a Clostridium sp. JS66 strain.

In another aspect, a problem to be solved by the present disclosure is to provide a method for producing biochemicals having excellent productivity through fermentation of a strain having improved carbon dioxide utilization ability.

In still another aspect, a problem to be solved by the present disclosure is to provide a method for producing biochemicals through fermentation of sugar without the generation of carbon dioxide.

### Technical Solution

To solve the problems, an embodiment of the present disclosure provides Clostridium sp. ALE-CO2 JS66 under accession number KCTC 15321BP.

An embodiment of the present disclosure provides a method for producing a metabolite having a carbon number of C2 to C6, the method comprising culturing Clostridium sp. ALE-CO2 JS66 under accession number KCTC 15321BP.

An embodiment of the present disclosure provides a method for fixing carbon, the method comprising: fixing carbon dioxide by culturing Clostridium sp. ALE-CO2 JS66 under accession number KCTC 15321BP by autotrophic fermentation in which the strain is cultured in a medium comprising a mixed gas comprising carbon dioxide and hydrogen, mixotrophic fermentation in which the strain is cultured by supplying hydrogen from the outside to a medium comprising sugar, thereby consuming even carbon dioxide derived from the sugar, or carbon-negative fermentation in which the strain is cultured by supplying hydrogen from the outside to a medium comprising sugar, thereby consuming carbon dioxide derived from the sugar and further continuously supplying carbon dioxide and hydrogen.

### Advantageous Effects

In an aspect, since the Clostridium sp. ALE-CO2 JS66 under accession number KCTC 15321BP, according to the present disclosure, has a significantly improved carbon dioxide utilization ability compared to a Clostridium sp. JS66 strain, which is a parent strain, carbon dioxide can be directly utilized as a carbon source to be converted into biochemicals. Further, an embodiment of the present disclosure is environmentally friendly and can efficiently produce high-value-added compounds from greenhouse gases comprising carbon dioxide or by-product gases from steel mills through a biological conversion process using the fermentation of the Clostridium sp. ALE-CO2 JS66.

### DESCRIPTION OF DRAWINGS

FIG. 1 is a schematic view schematically showing an adaptive laboratory evolution experiment process according to an embodiment of the present disclosure.

FIG. 2 is a graph illustrating an increased carbon dioxide (CO₂) consumption rate during an adaptive laboratory evolution step (ALE Round) for the preparation of Clostridium sp. ALE-CO2 JS66 according to an embodiment of the present disclosure.

FIG. 3 is a graph illustrating a comparison of carbon dioxide (CO₂) consumption rates between the initial and final stages (ALE Round 1 and 95) of adaptive laboratory evolution in the preparation step of Clostridium sp. ALE-CO2 JS66 according to an embodiment of the present disclosure.

FIG. 4 is a view illustrating a comparison of carbon dioxide (CO₂) consumption rates when Clostridium sp. ALE-CO2 JS66 (ALE-CO2) according to an embodiment of the present disclosure, a wild-type strain (wild-type), and a comparative example (ALE CO70) were subjected to autotrophic fermentation under the same conditions (supplying CO₂ and H₂ at 3:7 under 2 bar).

FIG. 5 is a view illustrating a comparison of hydrogen (H₂) consumption rates when Clostridium sp. ALE-CO2 JS66 (ALE-CO2) according to an embodiment of the present disclosure, a wild-type strain (wild-type), and a comparative example (ALE CO70) were subjected to autotrophic fermentation under the same conditions (supplying CO2 and H₂ at 3:7 under 2 bar).

FIG. 6 is a view illustrating a comparison of glucose consumptions when Clostridium sp. ALE-CO2 JS66 (ALE-CO2) according to an embodiment of the present disclosure, a wild-type strain (wild-type), and a comparative example (ALE CO70) were subjected to mixotrophic fermentation under the same conditions (supplying 5 g/L glucose and 1.5 bar H₂ gas).

FIG. 7 is a view illustrating a comparison of the consumptions of carbon dioxide (CO₂) derived from glucose when Clostridium sp. ALE-CO2 JS66 (ALE-CO2) according to an embodiment of the present disclosure, a wild-type strain (wild-type), and a comparative example (ALE CO70) were subjected to mixotrophic fermentation under the same conditions (supplying 5 g/L glucose and 1.5 bar H₂ gas).

FIG. 8 is a view illustrating a comparison of hydrogen (H₂) consumptions when Clostridium sp. ALE-CO2 JS66 (ALE-CO2) according to an embodiment of the present disclosure, a wild-type strain (wild-type), and a comparative example (ALE CO70) were subjected to mixotrophic fermentation under the same conditions (supplying 5 g/L glucose and 1.5 bar H₂ gas).

FIG. 9 is a view illustrating the production amount of metabolites (final products) by type of metabolite when Clostridium sp. ALE-CO2 JS66 (ALE-CO2) according to an embodiment of the present disclosure, a wild-type strain (WT), and a comparative example (ALE CO70) were subjected to mixotrophic fermentation under the same conditions (supplying 5 g/L glucose and 1.5 bar H₂ gas) (EtOH: ethanol, BuOH: butanol, HeOH: hexanol, AA: acetic acid, BA: butyric acid, and HA: hexanoic acid).

FIG. 10 is a graph illustrating metabolites produced by time periods during mixotrophic fermentation of Clostridium sp. ALE-CO2 JS66 (ALE-CO2) according to an embodiment of the present disclosure (EtOH: ethanol, BuOH: butanol, HeOH: hexanol, AA: acetic acid, BA: butyric acid, and HA: hexanoic acid).

FIG. 11 is a graph illustrating gas generation and consumption during heterotrophic fermentation under the conditions of 5 g/L glucose using Clostridium sp. ALE-CO2 JS66 (ALE-CO2) when an adsorption resin according to an embodiment of the present disclosure was used.

FIG. 12 is a graph illustrating gas generation and consumption during mixotrophic fermentation to supply hydrogen under the conditions of 5 g/L glucose using Clostridium sp. ALE-CO2 JS66 (ALE-CO2) when an adsorption resin according to an embodiment of the present disclosure was used.

FIG. 13 is a graph illustrating gas generation and consumption when mixotrophic fermentation was performed to supply hydrogen under the conditions of 5 g/L glucose using Clostridium sp. ALE-CO2 JS66 (ALE-CO2), and then carbon-negative fermentation was further performed to supply a mixed gas comprising carbon dioxide and hydrogen, when an adsorption resin according to an embodiment of the present disclosure was used.

FIG. 14 is a graph illustrating the amount of gas consumed when mixotrophic fermentation was performed to supply hydrogen under the conditions of 5 g/L glucose using Clostridium sp. ALE-CO2 JS66 (ALE-CO2), and then carbon-negative fermentation was further performed to supply a mixed gas comprising carbon dioxide and hydrogen, when an adsorption resin according to an embodiment of the present disclosure was used.

FIG. 15 is a view illustrating the production amount of metabolites (final products) by type of metabolite when heterotrophic fermentation was performed under the conditions of 5 g/L glucose using Clostridium sp. ALE-CO2 JS66 (ALE-CO2), when an adsorption resin according to an embodiment of the present disclosure was used (EtOH: ethanol, BuOH: butanol, HeOH: hexanol, AA: acetic acid, BA: butyric acid, and HA: hexanoic acid).

FIG. 16 is a view illustrating the production amount of metabolites (final products) by type of metabolite when mixotrophic fermentation was performed under the conditions of 5 g/L glucose using Clostridium sp. ALE-CO2 JS66 (ALE-CO2), when an adsorption resin according to an embodiment of the present disclosure was used (EtOH: ethanol, BuOH: butanol, HeOH: hexanol, AA: acetic acid, BA: butyric acid, and HA: hexanoic acid).

FIG. 17 is a view illustrating the production amount of metabolites (final products) by type of metabolite when carbon-negative fermentation was performed under the conditions of 5 g/L glucose using Clostridium sp. ALE-CO2 JS66 (ALE-CO2), when an adsorption resin according to an embodiment of the present disclosure was used (EtOH: ethanol, BuOH: butanol, HeOH: hexanol, AA: acetic acid, BA: butyric acid, and HA: hexanoic acid).

FIG. 18 is a graph illustrating a comparison of the increased production amounts of hexanoic acid when heterotrophic fermentation, mixotrophic fermentation, and carbon-negative fermentation were performed under the conditions of 5 g/L glucose using Clostridium sp. ALE-CO2 JS66 (ALE-CO2) when an adsorption resin according to an embodiment of the present disclosure was used.

FIG. 19 is a graph illustrating the gas partial pressure analyzed during fermentation in which hydrogen was introduced from the initial stage of culture under the conditions of 3 g/L glucose using Clostridium sp. ALE-CO2 JS66 (ALE-CO2), and the hydrogen partial pressure was maintained at 90 kPa or more while introducing 3 g/L glucose every 24 hours, when an adsorption resin according to an embodiment of the present disclosure was used.

FIG. 20 is a graph illustrating the glucose concentration analyzed during fermentation in which hydrogen was introduced from the initial stage of culture under the conditions of 3 g/L glucose using Clostridium sp. ALE-CO2 JS66 (ALE-CO2), and the hydrogen partial pressure was maintained at 90 kPa or more while introducing 3 g/L glucose every 24 hours, when an adsorption resin according to an embodiment of the present disclosure was used.

FIG. 21 is a graph illustrating the final product concentration analyzed during fermentation in which hydrogen was introduced from the initial stage of culture under the conditions of 3 g/L glucose using Clostridium sp. ALE-CO2 JS66 (ALE-CO2), and the hydrogen partial pressure was maintained at 90 kPa or more while introducing 3 g/L glucose every 24 hours, when an adsorption resin according to an embodiment of the present disclosure was used.

### BEST MODE FOR DISCLOSURE

Hereinafter, preferred examples of the present disclosure will be described in detail with reference to the accompanying drawings.

The examples of the present disclosure disclosed herein are exemplified for the purpose of describing the examples of the present invention only, and the examples of the present disclosure may be carried out in various forms and should not be construed to be limited to the examples described herein. Since the present disclosure may have various changes and different forms, it should be understood that the examples are not intended to limit the present disclosure to specific disclosure forms and they include all the changes, equivalents and replacements included in the spirit and technical scope of the present disclosure. Singular expressions include plural expressions unless the context clearly indicates otherwise. In the present application, the term “comprise”, “include” or “have” is intended to designate the existence of characteristics, numbers, steps, operations, constituent elements, and parts described in the specification or a combination thereof, and does not exclude in advance a possibility of the existence or addition of one or more other characteristics, numbers, steps, operations, constituent elements, and parts, or a combination thereof.

As used herein, the term “biochemical” refers to any chemical that can be obtained by fermentation of a microorganism. For example, the biochemical may include an organic acid such as acetic acid, butyric acid, and hexanoic acid, or an alcohol such as ethanol, butanol, and hexanol, or a chemical derived therefrom.

As used herein, the term “mixotrophic fermentation” refers to a fermentation method in which microorganisms are fermented in a medium comprising sugar, hydrogen gas is separately supplied from the outside to provide reducing power for fixing carbon dioxide produced from the sugar during fermentation, and the microorganisms consume both the sugar comprised in the medium and the carbon dioxide produced from the sugar.

An embodiment of the present disclosure may provide Clostridium sp. ALE-CO2 JS66.

The Clostridium sp. ALE-CO2 JS66 is a novel strain isolated and identified as having excellent carbon dioxide consumption ability, and was assigned the accession number KCTC15321BP on Feb. 9, 2023.

As an embodiment, the Clostridium sp. ALE-CO2 JS66 may produce metabolites using a mixed gas comprising carbon dioxide as a substrate. The Clostridium sp. ALE-CO2 JS66 may have a significantly improved ability to fix carbon dioxide and produce metabolites using carbon dioxide due to its excellent carbon dioxide consumption ability. As an embodiment, the metabolites have a carbon number of C2 to C6, specifically, may comprise one or more of organic acids and organic alcohols having a carbon number of C2 to C6, and more specifically, may comprise one or more of acetic acid, butyric acid, hexanoic acid, ethanol, butanol, and hexanol.

The Clostridium sp. ALE-CO2 JS66 according to an embodiment may be a novel strain that is improved by adaptive laboratory evolution of the wild-type strain Clostridium sp. JS66. In this case, as an embodiment, the Clostridium sp. JS66 may be a strain under accession number KCTC13355BP.

Strains belonging to the Clostridium genus have peripheral flagella as Gram-positive bacilli, and most of them are obligate anaerobic bacteria. Although the strains form endospores, the morphology and location of the strains vary depending on the bacteria. Clostridium sp. strains are a type of acetogen, and the acetogen naturally has the Wood-Ljungdahl pathway to fix carbon dioxide and produce acetyl-CoA using hydrogen as an electron donor. The acetyl-CoA is a precursor capable of producing biocompounds. However, a Clostridium sp. JS66 strain which is a wild-type strain, has a low carbon dioxide utilization ability, and thus, shows a slow growth rate when carbon dioxide is given as a sole carbon source, so that there is a limitation in which it is difficult to use the strain industrially.

Although the Clostridium sp. JS66 used as a parent strain in an embodiment of the present disclosure also generally produces acetic acid, butyric acid, hexanoic acid, ethanol, butanol, and hexanol as metabolites using carbon dioxide as a carbon source, the rate at which carbon dioxide is used is very slow due to the long lag phase, and the productivity of biocompounds is low because the consumption amount of carbon dioxide is also significantly small, so that it has been difficult to substantially use greenhouse gases comprising carbon dioxide or by-product gases from steel mills, and the like.

The Clostridium sp. ALE-CO2 JS66 according to the present disclosure is a novel strain genetically modified from the parent strain, shows a fast consumption rate when carbon dioxide is provided as a sole carbon source based on a significantly higher carbon dioxide utilization rate than the wild-type strain, and may exhibit a significantly high ability to utilize carbon dioxide generated from glucose and to produce metabolites (organic acids, organic alcohols) even during heterotrophic fermentation using glucose as a sugar source.

As an embodiment, the Clostridium sp. ALE-CO2 JS66 may be prepared by a preparation method comprising: (a) preparing Clostridium sp. ALE-CO70 JS66 by continuously sub-culturing Clostridium sp. JS66 in a medium comprising carbon monoxide as a carbon source to subject the Clostridium sp. JS66 to adaptive laboratory evolution (ALE) so as to be tolerant to carbon monoxide (CO), and (b) continuously sub-culturing the Clostridium sp. ALE-CO70 JS66 in a medium comprising carbon dioxide as a sole carbon source to subject the Clostridium sp. ALE-CO70 JS66 to adaptive laboratory evolution so as to improve its carbon dioxide utilization ability.

As an embodiment, when the sub-culture is continuously performed as described above in step (a), the carbon monoxide utilization of the strain may be improved to significantly reduce the lag phase. As an embodiment, adjusting the components and composition of the medium step by step may comprise gradually increasing the content of carbon monoxide comprised in the medium. For example, the content of carbon monoxide in each step may be a pressure percentage selected from among 30 pressure %, 40 pressure %, 50 pressure %, 60 pressure %, 70 pressure %, 80 pressure %, or 90 pressure % with respect to the total gas pressure, and may be adjusted such that the pressure percentage value increases as the step progresses. As an embodiment, the medium in the final step of adjusting the components and composition of the medium step by step may comprise a mixed gas comprising carbon monoxide, carbon dioxide, and hydrogen at a ratio of 7:1:2.

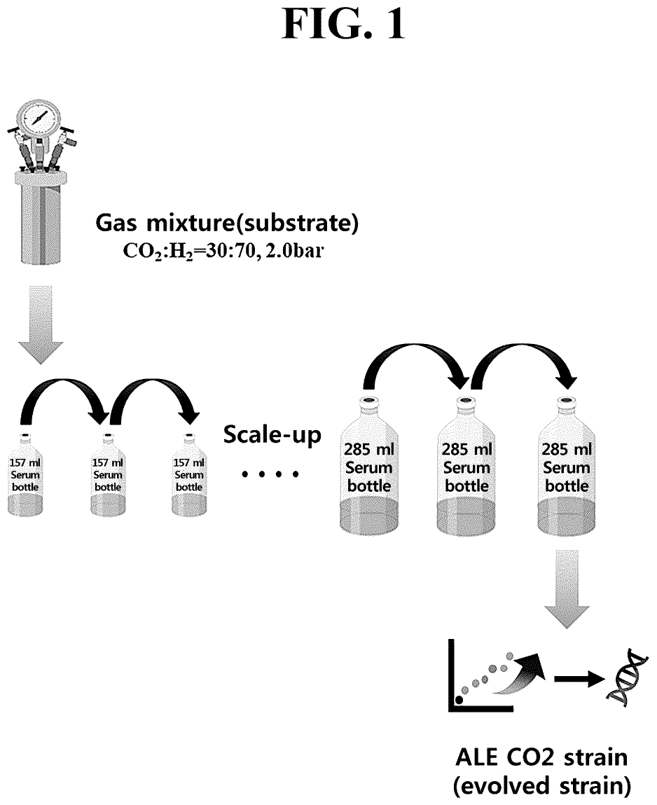

As an embodiment, the adaptive laboratory evolution step of (b) may comprise adjusting the components and composition of the medium step by step to adapt the strain to carbon dioxide, and may shorten the lag phase formed using carbon dioxide and prepare a strain having a carbon dioxide utilization rate significantly increased compared to those of Clostridium sp. JS66 and Clostridium sp. ALE-CO70 JS66. Specifically, culturing the strain in the adaptive laboratory evolution step may comprise continuously sub-culturing the strain one or more times in a medium having the same composition as the medium used in each step. When the strain is continuously sub-cultured as described above, the carbon dioxide utilization of the strain may be improved to significantly reduce the lag phase. As an embodiment, adjusting the components and composition of the medium step by step may comprise gradually increasing the content of carbon dioxide comprised in the medium. Specifically, the mixed gas comprising carbon dioxide and hydrogen gas comprised in the medium may comprise carbon dioxide and hydrogen at a ratio of 30:70. In this case, each step during the sub-culture may increase the content of carbon dioxide by adjusting the pressure of the mixed gas comprising carbon dioxide and hydrogen gas to 125 kPa, 150 kPa, and 200 kPa such that the pressure is increased as the step progresses. Further, an embodiment may comprise gradually decreasing the amount of microorganisms sub-cultured in each of the steps. For example, the amount of microorganisms sub-cultured in each of the steps may be adjusted such that for microorganisms corresponding to the log phase state, an inoculation amount is decreased to 4 ml, 3 ml, 2 ml, and 1 ml as the step progresses. In addition, an embodiment may gradually increase the volume of a head space during the culture in each of the steps. For example, the volume may be increased from 157 ml to 285 ml or from 137 ml to 265 ml. FIG. 1 schematically illustrates a method for strengthening the selection pressure by continuously sub-culturing cells one or more times in a new medium comprising a gas mixture when the cells reach the exponential or log phase, increasing the pressure of the gas mixture during the sub-culture, and adjusting the amount of cells to be inoculated and the volume of a sample bottle (serum bottle) in which the cells are cultured.

An embodiment of the present disclosure may provide a method for producing a metabolite having a carbon number of C2 to C6, the method comprising culturing the Clostridium sp. ALE-CO2 JS66.

In an embodiment, the method may simultaneously produce metabolites having a carbon number of C2 to C6. Specifically, the metabolites may comprise compounds or alcohols having a carbon number of C2 to C6. More specifically, the metabolites may comprise one or more of acetic acid, ethanol, butyric acid, butanol, hexanoic acid, and hexanol.

As an embodiment, the culturing step may comprise culturing Clostridium sp. ALE-CO2 JS66 in a medium comprising a gas comprising a carbon source as a substrate. Specifically, the gas comprising the carbon source may be a mixed gas comprising carbon dioxide and hydrogen, or carbon monoxide, carbon dioxide, and hydrogen. For example, the mixed gas may be a syngas that may be produced by a gasification process of coal, industrial waste, and the like.

As an embodiment, the culturing step may comprise subjecting Clostridium sp. ALE-CO2 JS66 to autotrophic fermentation in a medium comprising a gas comprising carbon dioxide as a substrate.

As another embodiment, the culturing step may comprise supplying a hydrogen-containing gas from the outside to a medium comprising sugar to perform mixotrophic fermentation using the sugar and carbon dioxide produced from the sugar as substrates.

As an embodiment, the sugar may comprise one or more of glucose, fructose, galactose, xylose, arabinose, mannose, cellobiose, and sucrose. As an embodiment, the concentration of the sugar is not limited, and the sugar may be comprised, for example, at a concentration of 1 g/L or more, 2 g/L or more, 3 g/L or more, 4 g/L or more, or 5 g/L or more with respect to the total volume of the medium. Specifically, the sugar may be comprised at a concentration of 5 g/L with respect to the total volume of the medium. In an embodiment, the strain may consume 90% or more, 95% or more, or 99% or more of the carbon dioxide produced from the sugar comprised in the medium.

As another embodiment, the culturing step may comprise supplying a hydrogen-containing gas from the outside to a medium comprising sugar to perform mixotrophic fermentation using the sugar and carbon dioxide produced from the sugar as substrates; and additionally supplying a gas mixture comprising carbon dioxide and hydrogen in addition to the carbon dioxide produced from the sugar to perform fermentation.

As still another embodiment, the culturing step may comprise a carbon-negative fermentation step in which the strain is cultured by supplying hydrogen from the outside to a medium comprising sugar, thereby consuming carbon dioxide derived from the sugar and further continuously supplying carbon dioxide and hydrogen.

As an embodiment, the method may further comprise recovering metabolites from the adsorption resin after the additionally supplying of the mixed gas to perform fermentation. In an embodiment of the present disclosure, an adsorption resin may be added to the medium as described above to remove hexanoic acid produced by the strain according to the present disclosure, thereby preventing the produced hexanoic acid itself from causing inhibition of hexanoic acid production due to cell membrane structural modification, pH reduction, and the like of acetogen strains. In addition, by adsorbing and removing metabolites that inhibit microorganisms, it is possible to provide a culture environment in which the microorganisms can continuously consume carbon dioxide without being inhibited.

As an embodiment, the medium may further comprise an adsorption resin of the metabolites. The adsorption resin is not limited as long as it can adsorb metabolites, and may comprise, for example, porous polymer particles. Specifically, the porous polymer particles may be capable of adsorbing substances having a molecular weight of 100 to 1000. Specifically, the porous polymer particles may comprise porous aromatic polymer particles. For example, the porous aromatic polymer particles may be porous polystyrene-based polymer particles. Specifically, the porous polymer particles may satisfy one or more of the followings: a specific surface area of 700 to 1500 m²/g; a pore radius of 40 to 85 Å; a particle size range of 200 to 1500 μm of the particles; and a pore volume of 0.8 to 1.8 ml/g. More specifically, the specific surface area may be 1100 to 1500 m²/g. More specifically, the pore radius may be 45 to 55 Å. More specifically, the particle size range of the particles may be 250 to 700 μm. More specifically, the pore volume may be 1.2 to 1.6 ml/g. As an embodiment, the adsorption resin may comprise one or more selected from the group consisting of TRILITE® GSH-20 (manufacturer: Samyang Corporation) and TRILITE® GSP-25 (manufacturer: Samyang Corporation).

An embodiment of the present disclosure may easily recover metabolites produced in the medium using the adsorption resin as described above in order to adsorb and remove the metabolites. Furthermore, by adsorbing and removing metabolites that inhibit microorganisms, it is possible to provide a culture environment in which the microorganisms can continuously consume carbon dioxide without being inhibited. Further, by facilitating the movement of syngas in the medium during the fermentation step, when an organic solvent or the like is used to remove metabolites through extraction, it is possible to solve a problem in that the organic solvent rises on the medium and the movement of the syngas is inhibited so that it cannot be used as a substrate.

As an embodiment, each of the cultures may be performed under the condition in which the initial total gas pressure of the gas is 3 bar or less, but is not limited thereto. Specifically, each of the cultures may be performed under the condition in which the initial total gas pressure of the gas is 2.5 bar or less, 2 bar or less, 1.5 bar or less, or 1 bar or less, and more than 0 bar.

In an embodiment, the culture temperature is not limited, but may be 25° C. to 37° C. in terms of increasing the production yield. Specifically, the culture temperature may be 25° C. or more, 26° C. or more, 27° C. or more, 28° C. or more, 29° C. or more, 30° C. or more, 31° C. or more, 32° C. or more, 33° C. or more, 34° C. or more, 35° C. or more, or 36° C. or more, and 37° C. or less, 36° C. or less, 35° C. or less, 34° C. or less, 33° C. or less, 32° C. or less, 31° C. or less, 30° C. or less, 29° C. or less, 28° C. or less, 27° C. or less, or 26° C. or less. For example, the culture may be performed at a temperature of 30° C.

In an embodiment, the culture may be performed under an initial pH condition of 5 to 8 in terms of increasing the production yield, and the pH may be changed as the culture progresses. For example, the culture may be performed under an initial pH condition of 5.5 to 7.

In an embodiment, the culture may be performed under anaerobic conditions. As used herein, the “anaerobic condition” refers to an environment in which the amount of oxygen is small to an extent that an obligate anaerobic microorganism can survive or oxygen is not present.

In an embodiment, the culture may be performed in a medium comprising a yeast extract. The medium may comprise a yeast extract at 1 g/L. In an embodiment, the culture may be performed in a medium comprising molybdenum and tungsten, which are known to be necessary for microbial gas culture. For example, the culture may be performed in a medium comprising one or more selected from a yeast extract, 2-N-morpholino ethane sulfonic acid (MES), potassium hydroxide (KOH), ammonium chloride (NH₄Cl), calcium chloride dihydrate (CaCl₂·2H₂O), magnesium sulfate heptahydrate (MgSO₄·7H₂O), sodium chloride (NaCl), potassium chloride (KCl), potassium phosphate (KH₂PO₄), potassium phosphate dibasic (K₂HPO₄), cysteine hydrochloride (HCl-cysteine), nitrilotriacetic acid, manganese sulfate hydrate (MnSO₄H₂O), ammonium sulfate ((NH₄)₂SO₄), ferrous sulfate heptahydrate (FeSO₄·7H₂O), ammonium ferrous sulfate hexahydrate (Fe(SO₄)₂ (NH₄)_{2·6}H₂O), cobalt chloride hexahydrate (CoCl₂·6H₂O), zinc sulfate heptahydrate (ZnSO₄·7H₂O), copper chloride dihydrate (CuCl₂·2H₂O), nickel chloride dihydrate (NiCl₂·2H₂O), sodium molybdate dihydrate (Na₂MoO₄·2H₂O), sodium tungstate (Na₂WO₄), aluminum potassium sulfate dodecahydrate (KAl(SO₄)₂·12H₂O), and boric acid (H₃BO₃).

In an embodiment, the method may further comprise, before the culture, performing seed culture in a medium comprising sugar. For example, the sugar may comprise one or more sugars of glucose, sucrose, fructose, galactose, xylose, arabinose, mannose, cellobiose, and sucrose, but is not limited thereto. For example, the sugar may comprise glucose. As an embodiment, the medium used for the seed culture may comprise sugar at, for example, 1 g/L to 10 g/L, for example 5 g/L.

The present disclosure may provide a method for fixing carbon, the method comprising fixing carbon dioxide by culturing the Clostridium sp. ALE-CO2 JS66 as an embodiment. Specifically, the method for fixing carbon may comprise fixing carbon dioxide by culturing the Clostridium sp. ALE-CO2 JS66 by autotrophic fermentation in which the strain is cultured in a medium comprising a mixed gas comprising carbon dioxide and hydrogen, mixotrophic fermentation in which the strain is cultured by supplying hydrogen from the outside to a medium comprising sugar, thereby consuming even carbon dioxide derived from the sugar, or carbon-negative fermentation in which the strain is cultured by supplying hydrogen from the outside to a medium comprising sugar, thereby consuming carbon dioxide derived from the sugar and further continuously supplying carbon dioxide and hydrogen. More specifically, the fixation method may be a method for fixing a syngas containing carbon dioxide and hydrogen comprised in greenhouse gases comprising carbon dioxide and/or by-product gases from steel mills.

### MODE FOR DISCLOSURE

Hereinafter, the present invention will be described in detail with reference to the Examples, Comparative Examples, and Test Examples. These are only provided for illustrative purposes in order to more specifically describe the present invention, and it will be obvious to a person with ordinary skill in the art that the scope of the present invention is not limited by the Examples, Comparative Examples, and Test Examples.

### [Preparation Example 1] Preparation of Clostridium sp. ALE-CO2 JS66

First, Clostridium sp. JS66 under accession number KCTC13355BP was prepared as a parent strain.

A medium for culturing the parent strain (P7-s) comprises 1 g of a yeast extract, 2 g of ammonium chloride (NH₄Cl), 0.08 g of calcium chloride (CaCl₂)₂H₂O), 0.4 g of magnesium sulfate (MgSO₄·7H₂O), 0.2 g of potassium chloride (KCl), 0.2 g of potassium phosphate (KH₂PO₄), 0.01 g of manganese sulfate (MnSO₄·H₂O), 0.002 g of sodium molybdate (NaMoO₄·2H₂O), 0.2 g of cysteine, and trace elements, per liter of total volume. The composition of the trace elements is as shown in the following Table 1.

TABLE 1

| Trace elements | mg/L |
|---|---|
| Nitrilotriacetic acid | 20 |
| MnSO₄•H₂O | 10 |
| Fe(SO₄)₂(NH₄)₂•6H₂O | 8 |
| CoCl₂•6H₂O | 2 |
| ZnSO₄•7H₂O | 0.002 |
| CuCl₂•2H₂O | 0.2 |
| NiCl₂•2H₂O | 0.2 |
| Na₂MoO₄•2H₂O | 0.2 |
| Na₂WO₄ | 0.2 |
| KAl(SO₄)₂•12H₂O | 0.2 |
| H₃BO₃ | 0.1 |

While fermentation was performed, 50 mM 2-[N-morpholino] ethanesulfonic acid (MES) was added for pH buffering, and the initial pH of the medium was adjusted to pH 6 using 1 M potassium hydroxide (KOH). Further, 2 g/L CaCO₃ was added to prevent a rapid decrease in pH. As a batch culture, 20 ml of the prepared medium was put into a 157 ml sample bottle (serum bottle), the parent strain was inoculated, and then when cells reached the log phase, the cells were continuously sub-cultured one or more times starting in an environment where a mixed gas comprising 30 pressure % carbon monoxide was supplied with respect to the total mixed gas pressure.

The sub-culture comprised a process in which the pressure ratio of carbon monoxide is sequentially increased to 30%, 50%, and 70% under a selection pressure. Specifically, the cells were sub-cultured under the condition of a mixed gas of total 1.5 bar (CO:Ar=3:7), and then sub-cultured under the condition of a mixed gas of total 1.5 bar (CO:CO₂:H₂=5:3:2). Thereafter, the sub-culture was performed under the condition of a mixed gas of total 1.5 bar (CO:CO₂:H₂=7:2:1) to obtain Clostridium sp. ALE-CO70 JS66.

Next, 20 ml of the medium (P7-s) was put into a separately prepared 157 ml sample bottle (serum bottle), the prepared Clostridium sp. ALE-CO70 JS66 was inoculated, and then when the cells reached the log phase, the cells were continuously sub-cultured. The sub-culture was performed by a batch culture method, and comprised a process of adjusting the pressure of the mixed gas, adjusting the amount of cells to be inoculated during sub-culture, and adjusting the volume of the sample bottle (serum bottle) in which the cells are cultured. Specifically, after sub-culture was performed under mixed gas (CO₂:H₂=30:70, 1.25 bar) conditions, sub-culture was performed under mixed gas (CO₂:H₂=30:70, 1.5 bar) conditions. Thereafter, sub-culture was performed under mixed gas (CO₂:H₂=30:70, 2.0 bar) conditions. During the sub-culture, the amount of cells to be inoculated was adjusted to 4, 2, and 1 ml. The volume of the sample bottle (serum bottle) in which the cells were cultured was adjusted from 157 ml to 285 ml. Finally, a culture solution was streak-plated on an agar medium to isolate a single colony, from which a novel microorganism having improved carbon dioxide consumption ability, Clostridium sp. ALE-CO2 JS66, was obtained.

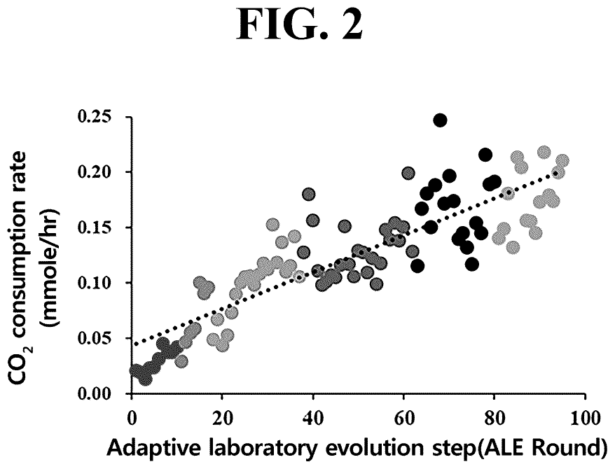

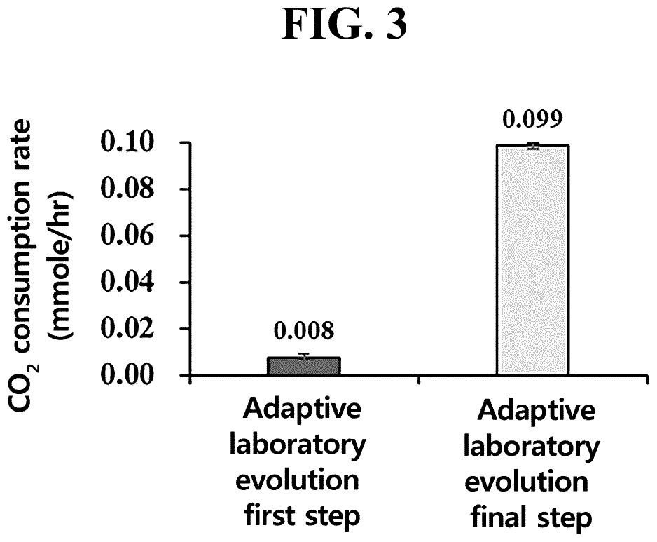

The consumption of the gas and the growth of the strain over time for the prepared Clostridium sp. ALE-CO2 JS66 were analyzed. The change in gas pressure over time was measured using a thermal conductivity detector (TCD) (Agilent Technology 6890N, USA). The column was Porapak Q, the inlet temperature was set to 100° C., the detector temperature was set to 200° C., and the mobile phase gas argon at 50° C. was set to 15.5 ml/min. The analysis of products such as ethanol, butanol, hexanol, acetic acid (AA), butyric acid (BA), and hexanoic acid (HA) were measured using a gas chromatograph (Agilent model 6890N gas chromatography). As a column used for the gas chromatograph, HP-INNOWax (Agilent, 30 m×0.32 mm×0.25 μm) was used, the inlet temperature was maintained at 250° C., the detector temperature was maintained at 250° C., and the oven temperature was increased by 10° C. from an initial temperature of 50° C. to a final temperature of 250° C. Helium gas was used as a carrier gas, and the pressure at the inlet was maintained at 9.41 psi. As a result, as illustrated in FIGS. 2 and 3, it was confirmed that the carbon dioxide consumption rate of Clostridium sp. ALE-CO2 JS66 rapidly evolved with each generation through adaptive laboratory evolution experiment techniques, and when the carbon dioxide consumption rates were compared between final step (ALE Round 95) and first step (ALE Round 1) of the adaptive laboratory evolution, the carbon dioxide consumption rate increased 12-fold or more from 0.008 mmol/hr to 0.099 mmol/hr.

### Test Example 1

In order to analyze the genetic modifications that occurred during the adaptive laboratory evolution process of Clostridium sp. ALE-CO2 JS66, the novel strain according to an embodiment of the present disclosure prepared in Preparation Example 1, the entire genome sequence was analyzed through next generation sequencing (NGS).

The analysis was performed using Illumina whole genome sequencing service provided by Macrogen, Inc., and the quality of the data was confirmed using FastQC. Annotation and alignment were performed using Breseq and NCBI BLAST to confirm genomic variations.

TABLE 2

| | | | | Amino | |
|---|---|---|---|---|---|
| | | Protein | | acid | |
| | | functional | | sequence | |
| | | classification | | change | |
| | Gene location | (eggNOG | | (AA | Gene function |
| | (Locus tag) | Category) | Mutation | change) | (Description) |
| 1 | CCP7_02282 | S | Intergenic (−59) | — | Chemotaxis |
| | | | | | protein CheY |
| 2 | CCP7_03258 | K | V159F (GTC->TTC) | Val159Phe | Transcription |
| | | | | | regulator, LysR |
| | | | | | family |
| 3 | CCP7_03258 | K | N37K (AAC->AAA) | Asn37Lys | Transcription |
| | | | | | regulator, LysR |
| | | | | | family |
| 4 | CCP7_04074 | C | Intergenic (−294) | — | Hydrogenase large |
| | | | | | subunit domain |
| | | | | | protein |
| 5 | CCP7_05528 | E | Intergenic (−79) | — | Glutamate/gamma- |
| | | | | | aminobutyrate |
| | | | | | family transporter |
| | | | | | YjeM |
| 6 | CCP7_05670 | G | E337* (GAG->TAG | Glu337X | Rhamnulokinase |
| 7 | CCP7_01065 | C | 41 base pair | — | NADH: ubiquinone |
| | | | insertion | | reductase (H(+)- |
| | | | | | translocating) |

As shown in Table 2, when the wild-type strain, which had not been subjected to an adaptive laboratory evolution step for carbon dioxide, was compared with Clostridium sp. ALE-CO70 JS66, a total of seven mutations occurred in the Clostridium sp. ALE-CO2 JS66, and from this, it could be confirmed that Clostridium sp. ALE-CO2 JS66 is a novel microorganism which is genetically different from the wild-type strain and Clostridium sp. ALE-CO70 JS66 and has different characteristics from those of the strains.

### Test Example 2

The following experiment was performed to confirm the improved carbon dioxide fixation ability of the microorganism according to the present disclosure.

First, a 25% glycerol cell stock prepared from the Clostridium sp. ALE-CO2 JS66 of Preparation Example 1 using a 99% glycerol stock reagent (manufacturer: Junsei Chemical Co., Ltd.) was seed-cultured. A medium for the seed culture was prepared by adding 5 g of glucose to a medium (mP2) comprising 6.0 g of a yeast extract, 0.2 g of magnesium sulfate, 0.01 g of manganese sulfate, 0.01 g of ferrous sulfate, 0.01 g of sodium chloride, 0.5 g of potassium phosphate monobasic (KH₂PO₄), and 0.5 g of potassium phosphate dibasic (K₂HPO₄) per liter of total volume, and after 20 mL of the medium was aliquoted into each 57 mL sample bottle (serum bottle), the gas in each medium was substituted with argon gas, and the sample bottle was sealed with a rubber stopper and aluminum seal, and then sterilized at 121° C. and 1.5 atm for 15 minutes for use in the experiment.

Next, the seed-cultured Clostridium sp. ALE-CO2 JS66 was inoculated into the same P7-s medium as the medium used in Preparation Example 1 as the main culture, and then a mixed gas consisting of carbon dioxide (CO₂) and hydrogen (H₂) at a ratio of 3:7 was supplied at a total pressure of 2 bar. The carbon dioxide consumption rate was confirmed by performing autotrophic fermentation in a shaking incubator while culturing the strain at a temperature of 30° C. and a rotation speed of 200 rpm.

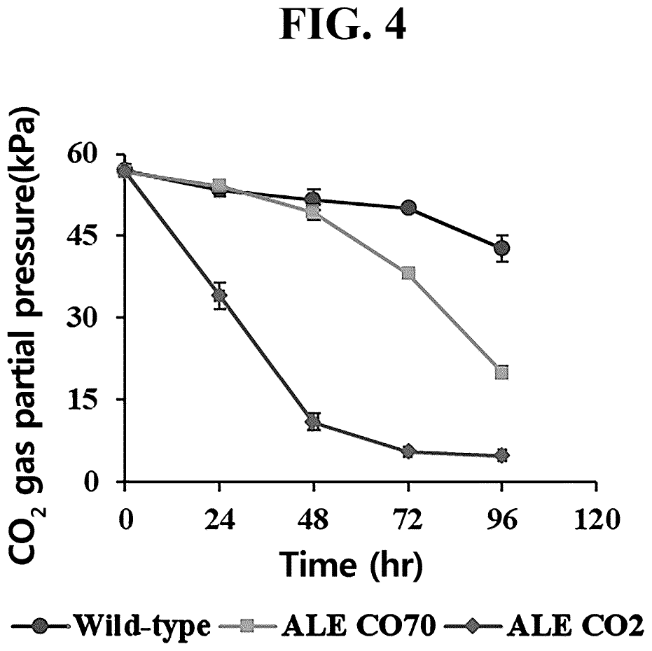

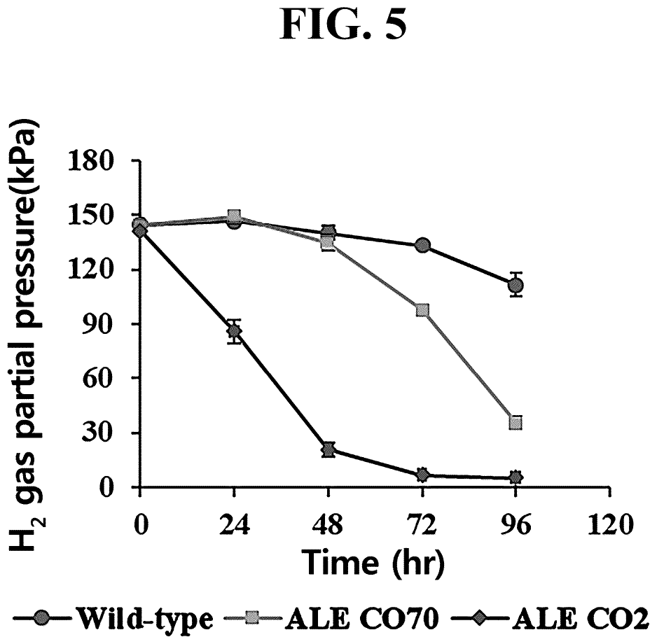

Clostridium sp. JS66 (wild-type) and the comparative example Clostridium sp. ALE-CO70 JS66 (ALE-CO70) were each subjected to autotrophic fermentation under the same conditions and methods as described above, the carbon dioxide consumption rates thereof were compared with that of Clostridium sp. ALE-CO2 JS66 (ALE-CO2), and the results are illustrated together in FIGS. 4 and 5.

As a result, Clostridium sp. ALE-CO2 JS66 (ALE-CO2), the novel strain according to the present disclosure, consumed carbon dioxide at a fast rate in the initial stage without any lag phase compared to Clostridium sp. JS66 (wild-type) and Clostridium sp. ALE-CO70JS66 (ALE-CO70). This indicates that the novel strain according to the present disclosure can be cultured while maintaining and preserving its improved carbon dioxide utilization ability.

### Test Example 3

In order to confirm whether fermentation without the generation of carbon dioxide is possible through the culture tendency of consuming carbon dioxide generated during sugar fermentation through mixotrophic fermentation in which hydrogen is supplied as an electron donor from the outside during glucose fermentation by the Clostridium sp. ALE-CO2 JS66 (ALE-CO2) microorganism according to the present disclosure, the following experiment was performed.

First, a 25% glycerol cell stock prepared from the Clostridium sp. ALE-CO2 JS66 of Preparation Example 1 using a 99% glycerol stock reagent (manufacturer: Junsei Chemical Co., Ltd.) was seed-cultured. A medium for the seed culture was prepared by adding 5 g of glucose to a medium (mP2) comprising 1.0 g of a yeast extract, 0.2 g of magnesium sulfate, 0.01 g of manganese sulfate, 0.01 g of ferrous sulfate, 0.01 g of sodium chloride, 0.5 g of potassium phosphate monobasic (KH₂PO₄), and 0.5 g of potassium phosphate dibasic (K₂HPO₄) per liter of total volume, and after 20 mL of the medium was aliquoted into each 57 mL sample bottle (serum bottle), the gas in each medium was substituted with argon gas, and the sample bottle was sealed with a rubber stopper and aluminum seal, and then sterilized at 121° C. and 1.5 atm for 15 minutes for use in the experiment.

Next, the seed-cultured Clostridium sp. ALE-CO2 JS66 was inoculated onto a medium (mP7-s) prepared by adding 5 g/L glucose as a sugar source to the P7-s medium which is the same as that used in Preparation Example 1 as the main culture, and then a gas consisting of a proportion of 100% hydrogen (H₂) was supplied at a total pressure of 1.5 bar in order to provide reducing power for fixing carbon dioxide released from the sugar, and then mixotrophic fermentation was performed in a shaking incubator at a temperature of 30° C. and a rotation speed of 200 rpm using the gas.

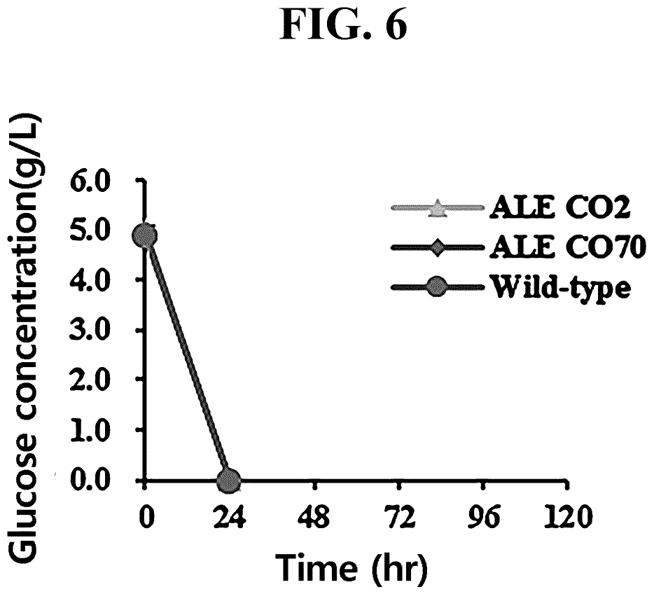

Clostridium sp. JS66 (wild-type) and the comparative example Clostridium sp. ALE-CO70 JS66 (ALE-CO70) were each subjected to mixotrophic fermentation under the same conditions and methods as described above, the carbon dioxide consumption rates thereof were compared with that of Clostridium sp. ALE-CO2 JS66 (ALE-CO2), and the results are illustrated together in FIGS. 6, 7, and 8.

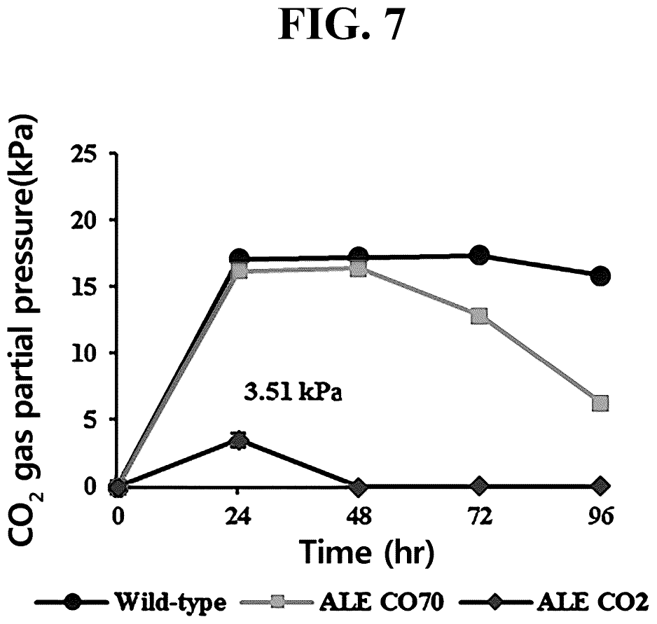

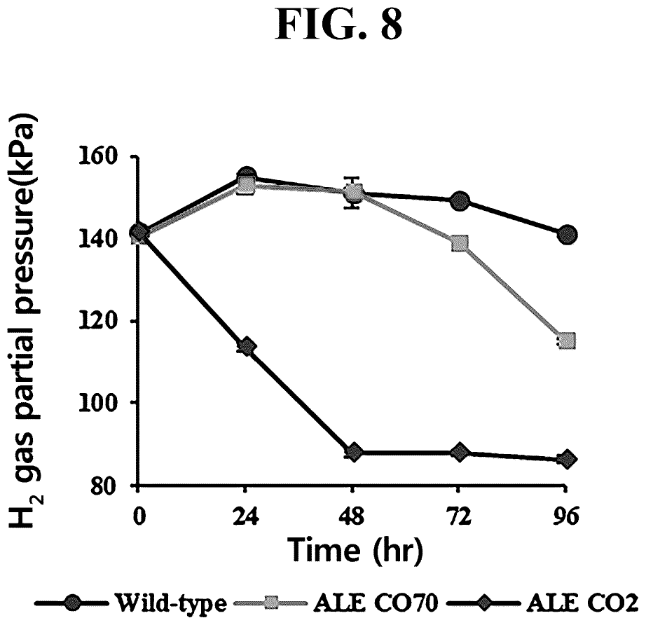

As a result, all of the Clostridium sp. ALE-CO2 JS66 (ALE-CO2) which is the novel strain according to the present disclosure, Clostridium sp. JS66 (wild-type), and Clostridium sp. ALE-CO70 JS66 (ALE-CO70) simultaneously generated carbon dioxide (FIG. 7) while consuming 5 g/L glucose within 24 hours (FIG. 6), but only Clostridium sp. ALE-CO2 JS66 (ALE-CO2), the novel strain according to the present disclosure, consumed all of the generated carbon dioxide within 48 hours, and also consumed 55.32 kPa of hydrogen required for carbon dioxide based on 48 hours (FIG. 8). Only Clostridium sp. ALE-CO2 JS66, the novel strain according to the present disclosure, implemented sugar fermentation without the generation of carbon dioxide while consuming all the carbon dioxide generated from sugar within 48 hours, and was able to achieve total carbon utilization without loss as carbon dioxide by utilizing and fixing all the carbon comprised in glucose by the microorganism.

This means that Clostridium sp. ALE-CO2 JS66, the novel strain according to the present disclosure, is not a strain that has excellent carbon dioxide utilization ability only in the presence of carbon dioxide as a sole carbon source, but is a strain that has sugar consumption ability and carbon dioxide utilization ability at the same time.

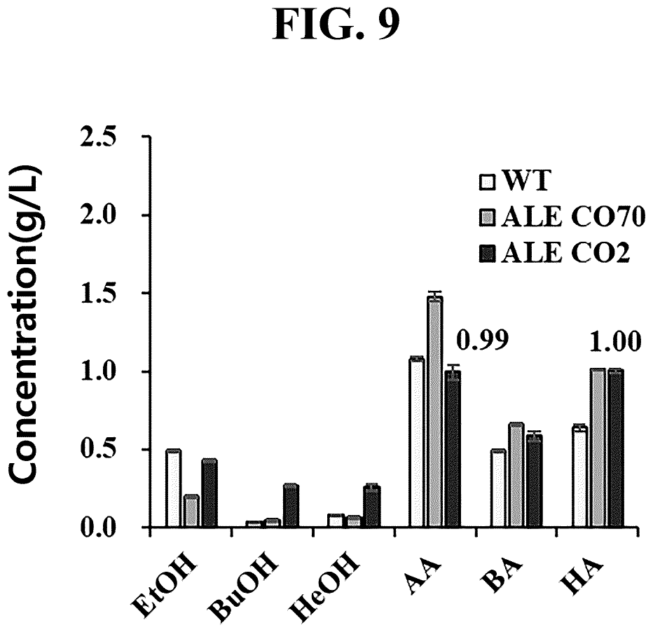

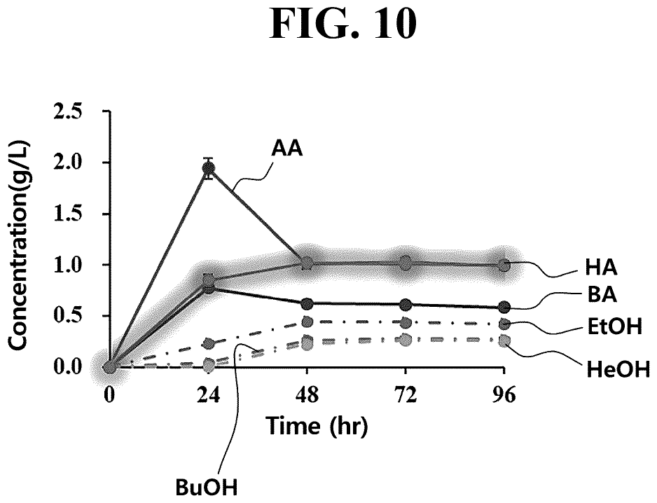

FIGS. 9 and 10 are views analyzing and showing the production amount of metabolites produced through the mixotrophic fermentation as described above and the production change over fermentation time. From FIG. 9, it can be confirmed that along with the production of hexanol, butanol, and hexanoic acid (HA) through sugar fermentation without the generation of carbon dioxide by Clostridium sp. ALE-CO2 JS66 (ALE-CO2), the novel strain according to the present disclosure, the overall production of biochemicals having a carbon number of C2 to C6 is increased. In addition, from FIG. 10, it can be confirmed that when the novel strain, Clostridium sp. ALE-CO2 JS66 (ALE-CO2), consumes all the amount of glucose given and then consumes the carbon dioxide generated and the hydrogen supplied from the outside, the concentration of acetic acid is decreased while acetic acid is consumed, and the concentrations of a C6 organic acid hexanoic acid, a C6 alcohol hexanol, and a C4 alcohol butanol are increased, resulting in increased production of the substances. This means that the novel strain Clostridium sp. ALE-CO2 JS66 (ALE-CO2) shows improved characteristics not only in the production of C2 compounds but also in the production of C4 to C6 compounds through chain elongation.

### Test Example 4

The following experiment was performed in order to compare the consumptions of carbon dioxide of Clostridium sp. ALE-CO2 JS66, the novel strain according to the present disclosure, according to the fermentation method.

To remove hexanoic acid, which inhibits microbial growth, in situ, an adsorption resin TRILITE® GSP-25 (manufacturer: Samyang Corporation) was injected into a medium and used. The adsorption resin was used in an amount of 80 g per liter of total medium volume.

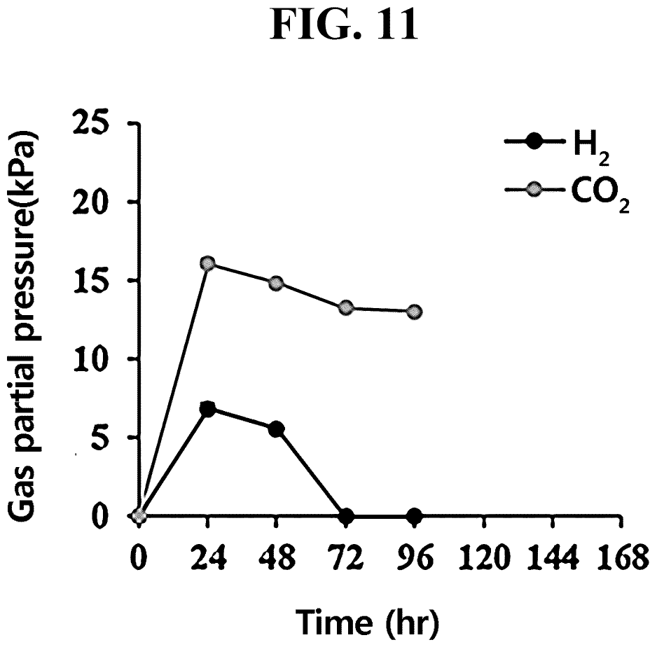

First, heterotrophic fermentation was performed in the same manner as in the mixotrophic fermentation described in Test Example 3, except that hydrogen gas was not supplied from the outside, and the change in gas partial pressure over fermentation time is illustrated in FIG. 11.

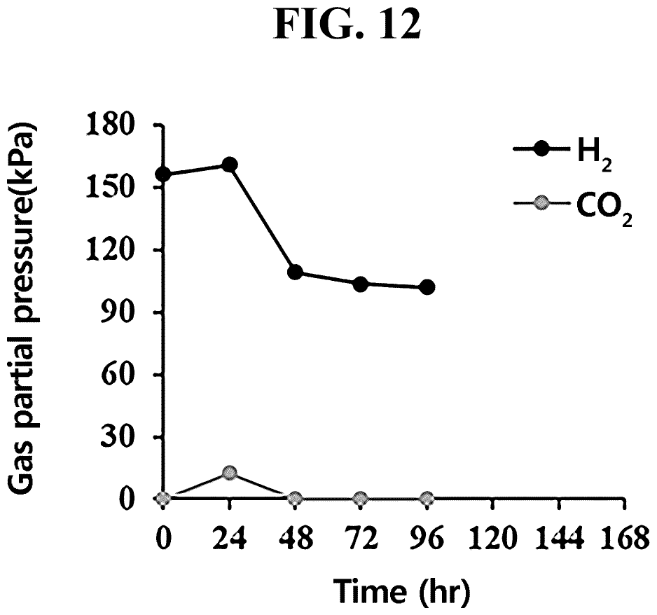

FIG. 12 illustrates the change in gas partial pressure over fermentation time when hydrogen gas was supplied from the outside and mixotrophic fermentation was performed in the same manner as in Test Example 3.

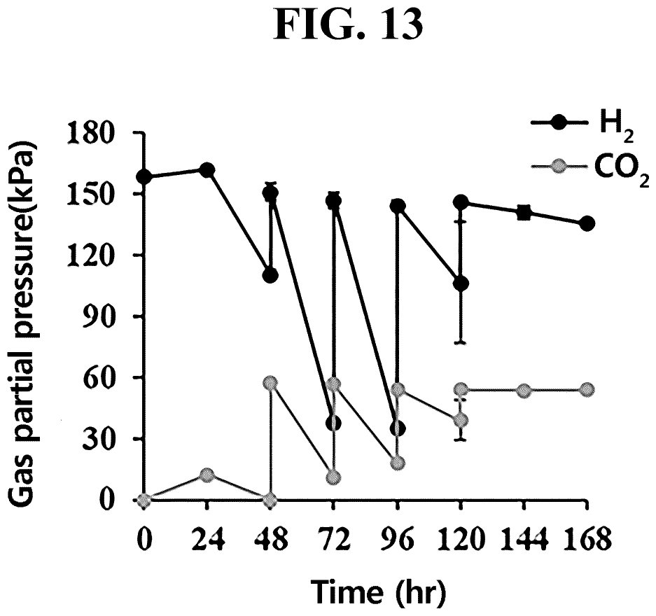

Finally, fermentation was performed in the same manner as the above mixotrophic fermentation, but after all the carbon dioxide was consumed, carbon-negative fermentation was performed by further supplying an additional gas mixture consisting of carbon dioxide (CO₂) and hydrogen (H₂) at a ratio of 3:7 at a total pressure of 1.5 bar four times, and the change in gas partial pressure over fermentation time is illustrated in FIG. 13.

After the culture was completed, each of the adsorbed ethanol, butanol, hexanol, acetic acid, butyric acid, and hexanoic acid was recovered from the separated adsorption resins. Specifically, 10 ml of ethyl acetate was added to each of the separated adsorption resins, and the resulting mixture was reacted in a shaking incubator at 30° C. and 150 rpm for 3 hours to recover each substance. Next, the products remaining in the ethyl acetate-based solution comprising the recovered substances and the culture solution which was not adsorbed by the adsorption resin were analyzed. Ethanol, butanol, hexanol, acetic acid (AA), butyric acid (BA), and hexanoic acid (HA) were analyzed using a gas chromatography (Agilent Technologies 6890N, CA, USA) equipped with a flame ionization detector (FID). As a column, HP-INNOWax (Agilent, 30 m×0.32 mm×0.25 μm) was used, the inlet temperature was maintained at 25° C., the detector temperature was maintained at 250° C., and the oven temperature was increased by 10° C./min from an initial temperature of 50° C. to a final temperature of 250° C. Helium gas was used as a carrier gas, and the pressure at the inlet was maintained at 9.41 psi.

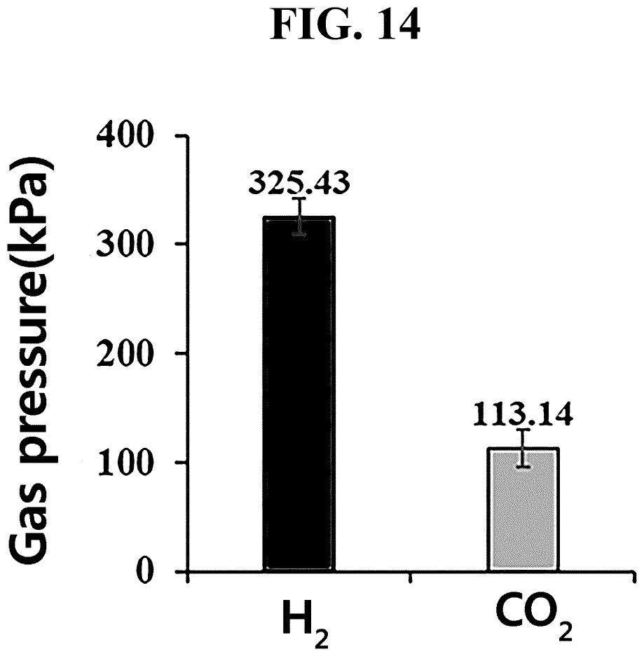

As a result, it can be confirmed that Clostridium sp. ALE-CO2 JS66 (ALE-CO2), the novel strain according to the present disclosure, consumes the additionally provided gas mixture at a fast rate in a 24-hour unit during carbon-negative fermentation compared to heterotrophic fermentation and mixotrophic fermentation, thereby showing more improved fermentation efficiency. FIG. 14 illustrates the amount of hydrogen and carbon dioxide additionally consumed during carbon-negative fermentation.

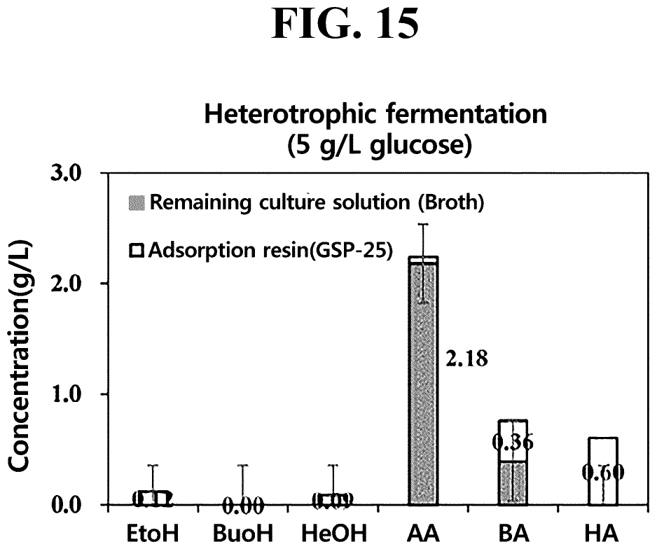

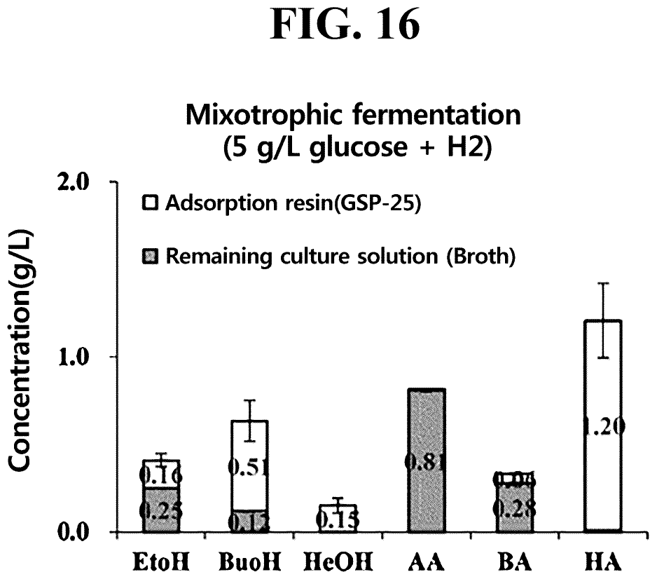

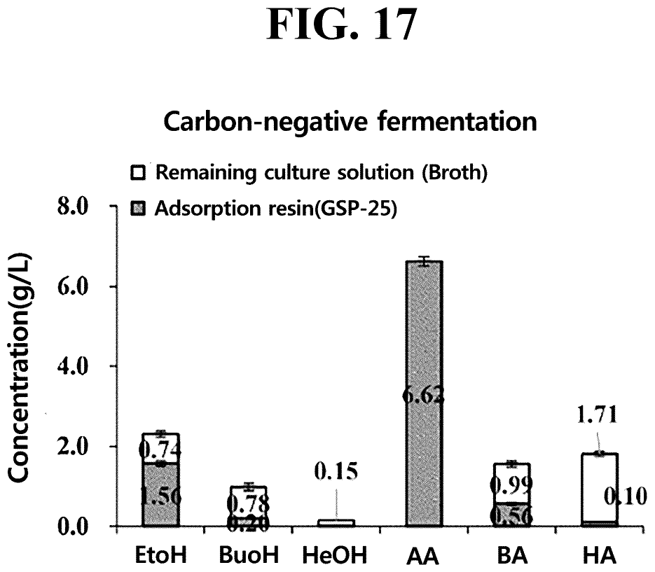

FIGS. 15 to 17 illustrate the final products during heterotrophic fermentation, mixotrophic fermentation, and carbon-negative fermentation, respectively, and it can be confirmed that the production of alcohols and the production of C4 to C6 compounds were increased during mixotrophic fermentation to which additional hydrogen was added and carbon-negative fermentation.

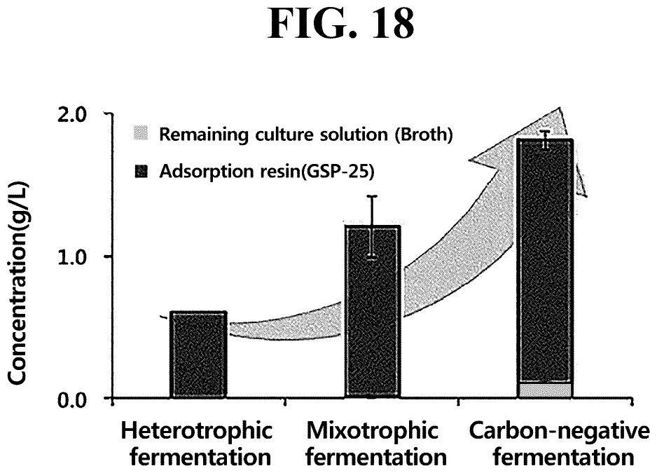

FIG. 18 illustrates a comparison of hexanoic acid production during heterotrophic fermentation, mixotrophic fermentation, and carbon-negative fermentation, and it can be confirmed that the production amount during carbon-negative fermentation showed 1.71 g/L compared to the production amount (1.20 g/L) during mixotrophic fermentation, indicating a 1.4-fold increase in hexanoic acid production during carbon-negative fermentation.

### Test Example 5

The following experiment was performed in order to examine the consumption of carbon dioxide of Clostridium sp. ALE-CO2 JS66, the novel strain according to the present disclosure, according to the additional introduction of sugar.

To remove hexanoic acid, which inhibits microbial growth, in situ, an adsorption resin TRILITE® GSP-25 (manufacturer: Samyang Corporation) was injected into a medium and used. The adsorption resin was used in an amount of 80 g per liter of total medium volume.

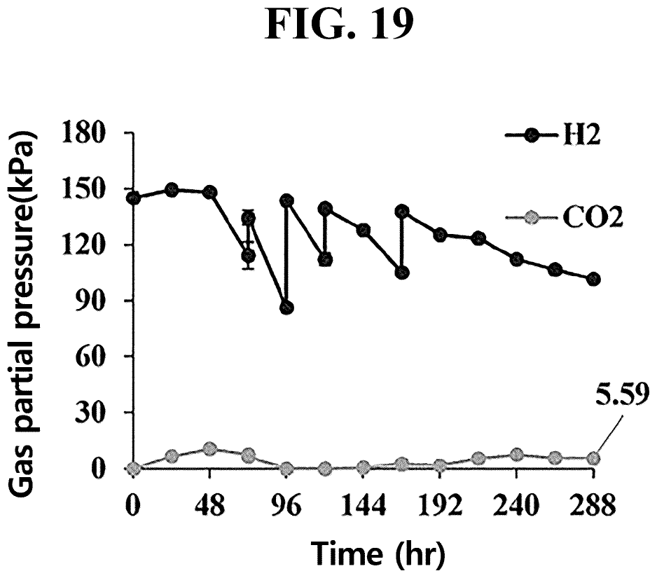

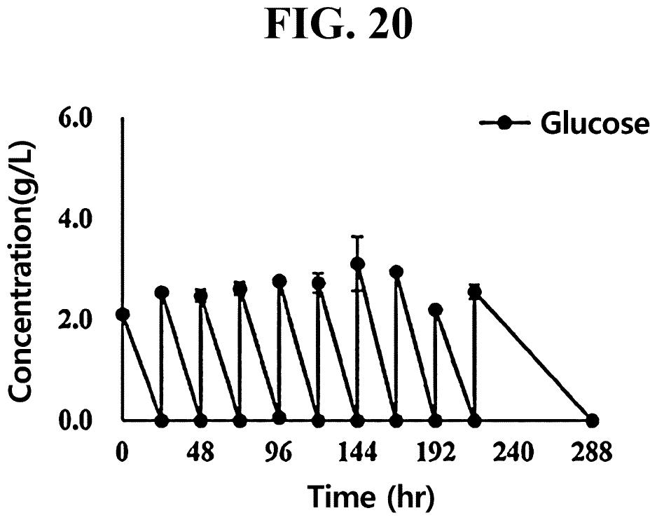

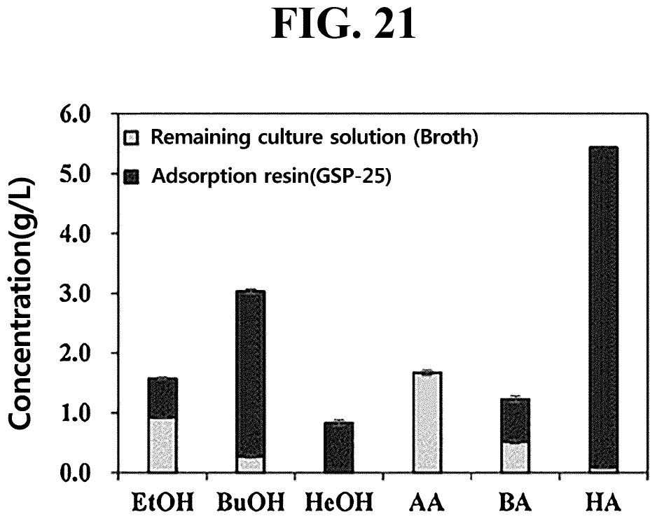

Fermentation was performed in the same manner as in the mixotrophic fermentation described in Test Example 3, while introducing 3 g/L glucose and hydrogen from the beginning, but additionally introducing 3 g/L glucose every 24 hours, and introducing hydrogen to maintain 90 kPa or more. The change in gas partial pressure over fermentation time is illustrated in FIG. 19. The glucose consumption tendency is illustrated in FIG. 20, and the final products were analyzed in the same manner as in Test Example 4, and are illustrated in FIG. 21.

As a result, Clostridium sp. ALE-CO2 JS66 (ALE-CO2), the novel strain according to the present disclosure, simultaneously consumed glucose introduced every 24 hours, hydrogen, and carbon dioxide derived and generated from the sugar, and thus, showed low carbon dioxide partial pressure characteristics (FIG. 19). At the end of fermentation, a total of 25 g/L glucose had been consumed (FIG. 20), and the final carbon dioxide generation was only 5.6 KPa (FIG. 19). That is, ALE-CO2 JS66 (ALE-CO2) exhibited the characteristics of maintaining the carbon dioxide consumption ability while continuously consuming sugar. From FIG. 21, it can be confirmed that as the products, the proportion of C4 to C6 compounds (BuOH, HeOH, BA, and HA) is higher than that of the C2 compounds ethanol (EtOH) and acetate (AA), and particularly, the production of hexanoic acid (HA) is high.

The present disclosure may provide the following embodiments as an example.

### First Embodiment

Clostridium sp. ALE-CO2 JS66 under accession number KCTC 15321BP.

### Second Embodiment

The Clostridium sp. ALE-CO2 JS66 of First Embodiment, wherein metabolites are produced using carbon dioxide as a carbon source.

### Third Embodiment

The Clostridium sp. ALE-CO2 JS66 of First or Second Embodiment, wherein metabolites are produced using a mixed gas comprising carbon dioxide and hydrogen as a substrate.

### Fourth Embodiment

The Clostridium sp. ALE-CO2 JS66 of Second or Third Embodiment, wherein the metabolites are one or more of organic acids and organic alcohols having a carbon number of C2 to C6.

### Fifth Embodiment

The Clostridium sp. ALE-CO2 JS66 of Third Embodiment, wherein the substrate further comprises carbon monoxide.

### Sixth Embodiment

The Clostridium sp. ALE-CO2 JS66 of Fourth Embodiment, wherein the metabolites comprise one or more of acetic acid, ethanol, butyric acid, butanol, hexanoic acid, and hexanol.

### Seventh Embodiment

A method for producing a metabolite having a carbon number of C2 to C6, the method comprising culturing the Clostridium sp. ALE-CO2 JS66 of any one of First to Sixth Embodiments.

### Eighth Embodiment

The method of Seventh Embodiment, wherein the culturing step comprises culturing the Clostridium sp. ALE-CO2 JS66 in a medium comprising a gas comprising a carbon source as a substrate.

### Ninth Embodiment

The method of Eighth Embodiment, wherein the gas comprising the carbon source is a mixed gas comprising carbon dioxide and hydrogen, or carbon monoxide, carbon dioxide, and hydrogen.

### Tenth Embodiment

The method of any one of Seventh to Ninth Embodiments, wherein the culturing step comprises performing autotrophic fermentation in a medium comprising a gas comprising carbon dioxide as a substrate.

### Eleventh Embodiment

The method of any one of Seventh to Tenth Embodiments, wherein the culturing step comprises supplying a hydrogen-containing gas from the outside to a medium comprising sugar to perform mixotrophic fermentation using the sugar and carbon dioxide produced from the sugar as substrates.

### Twelfth Embodiment

The method of Eleventh Embodiment, wherein the sugar comprises one or more of glucose, fructose, galactose, xylose, arabinose, mannose, cellobiose, and sucrose.

### Thirteenth Embodiment

The method of any one of Seventh to Twelfth Embodiments, wherein the culturing step further comprises additionally supplying sugar to the medium.

### Fourteenth Embodiment

The method of any one of Seventh to Thirteenth Embodiments, wherein the culturing step further comprises additionally supplying a mixed gas comprising carbon dioxide and hydrogen in addition to the carbon dioxide produced from the sugar to perform fermentation, wherein the medium further comprises an adsorption resin of metabolites.

### Fifteenth Embodiment

The method of Fourteenth Embodiment, further comprising recovering metabolites from the adsorption resin after the additionally supplying of the mixed gas to perform fermentation.

### Sixteenth Embodiment

The method of any one of Seventh to Fifteenth Embodiments, wherein the metabolites comprise one or more of acetic acid, ethanol, butyric acid, butanol, hexanoic acid, and hexanol.

### Seventeenth Embodiment

A method for fixing carbon, the method comprising: fixing carbon dioxide by culturing the Clostridium sp. ALE-CO2 JS66 under accession number KCTC 15321BP of any one of First to Sixth Embodiments by autotrophic fermentation in which the strain is cultured in a medium comprising a mixed gas comprising carbon dioxide and hydrogen, mixotrophic fermentation in which the strain is cultured by supplying hydrogen from the outside to a medium comprising sugar, thereby consuming even carbon dioxide derived from the sugar, or carbon-negative fermentation in which the strain is cultured by supplying hydrogen from the outside to a medium comprising sugar, thereby consuming carbon dioxide derived from the sugar and further continuously supplying carbon dioxide and hydrogen.

### [Accession Number]

Name of depository institution: Korean Collection for Type Cultures (KCTC), Biological Resource Center
Accession number: KCTC15321BP
Date of deposit: 20230209

### STATEMENT REGARDING PRIOR DISCLOSURES BY THE INVENTOR OR A JOINT INVENTOR

The inventors of the present application have made a related disclosure in Tae Kwan MOON et al., “Adaptive Laboratory Evolution (ALE) of Clostridium sp. JS66 for Enhancing CO2 fixation,” 2022 KSBB Fall Meeting and International Symposium, on Apr. 15, 2022. The related disclosure was made less than one year before the effective filing date (Apr. 13, 2023) of the present application. The present application names two (Tae Kwan MOON and Youngsoon UM) of the authors of the disclosure as joint inventors and omits the other two authors (Hyun ju OH and Sung Ok HAN) of the disclosure, because these omitted authors of the disclosure did not make contribution to conception of the invention. Accordingly, the related disclosure is a grace period inventor-originated disclosure, and thus is disqualified from prior art under 35 U.S.C § 102(a)(1) against the present application. See 35 U.S.C § 102(b)(1)(A).

1. Clostridium sp. ALE-CO2 JS66 under accession number KCTC 15321BP.

2. The Clostridium sp. ALE-CO2 JS66 according to claim 1, which produces metabolites using carbon dioxide as a carbon source.

3. The Clostridium sp. ALE-CO2 JS66 according to claim 1, which produces metabolites using a mixed gas comprising carbon dioxide and hydrogen as a substrate.

4. The Clostridium sp. ALE-CO2 JS66 according to claim 2, wherein the metabolites are one or more of organic acids and organic alcohols having a carbon number of C2 to C6.

5. The Clostridium sp. ALE-CO2 JS66 according to claim 3, wherein the substrate further comprises carbon monoxide.

6. The Clostridium sp. ALE-CO2 JS66 according to claim 4, wherein the metabolites comprise one or more of acetic acid, ethanol, butyric acid, butanol, hexanoic acid, and hexanol.

7. A method for producing a metabolite having a carbon number of C2 to C6, the method comprising culturing the Clostridium sp. ALE-CO2 JS66 of claim 1.

8. The method of claim 7, wherein the culturing step comprises culturing the Clostridium sp. ALE-CO2 JS66 in a medium comprising a gas comprising a carbon source as a substrate.

9. The method of claim 8, wherein the gas comprising the carbon source is a mixed gas comprising carbon dioxide and hydrogen, or carbon monoxide, carbon dioxide, and hydrogen.

10. The method of claim 7, wherein the culturing step comprises performing autotrophic fermentation in a medium comprising a gas comprising carbon dioxide as a substrate.

11. The method of claim 7, wherein the culturing step comprises supplying a hydrogen-containing gas from an outside to a medium comprising sugar to perform mixotrophic fermentation using the sugar and carbon dioxide produced from the sugar as substrates.

12. The method of claim 11, wherein the sugar comprises one or more of glucose, fructose, galactose, xylose, arabinose, mannose, cellobiose, and sucrose.

13. The method of claim 7, wherein the culturing step further comprises additionally supplying sugar to the medium.

14. The method of claim 7, wherein the culturing step further comprises additionally supplying a mixed gas comprising carbon dioxide and hydrogen in addition to the carbon dioxide produced from the sugar, and
wherein the medium further comprises an adsorption resin of metabolites.

15. The method of claim 14, further comprising recovering metabolites from the adsorption resin after the additionally supplying of the mixed gas to perform fermentation.

16. The method of claim 7, wherein the metabolites comprise one or more of acetic acid, ethanol, butyric acid, butanol, hexanoic acid, and hexanol.

17. A method for fixing carbon, the method comprising: fixing carbon dioxide by culturing the Clostridium sp. ALE-CO2 JS66 under accession number KCTC 15321BP of claim 1 by autotrophic fermentation in which the strain is cultured in a medium comprising a mixed gas comprising carbon dioxide and hydrogen, mixotrophic fermentation in which the strain is cultured by supplying hydrogen from an outside to a medium comprising sugar, thereby consuming even carbon dioxide derived from the sugar, or carbon-negative fermentation in which the strain is cultured by supplying hydrogen from an outside to a medium comprising sugar, thereby consuming carbon dioxide derived from the sugar and additionally introducing carbon dioxide and hydrogen to consume even carbon dioxide.
